(19) United States
(12) Patent Application Publication
(10) Pub. No.: US 2015/0173818 A1
(43) Pub. Date: Jun. 25, 2015

(54) MINIMALLY INVASIVE METHOD AND APPLICATIONS FOR INJECTIBLE MATERIALS

(71) Applicants: Gamal Baroud, Quebec (CA); Stephan Becker, Vienna (AT); Denis Imbeault, Quebec (CA)

(72) Inventors: Gamal Baroud, Quebec (CA); Stephan Becker, Vienna (AT); Denis Imbeault, Quebec (CA)

(21) Appl. No.: 14/636,923

(22) Filed: Mar. 3, 2015

Related U.S. Application Data
(63) Continuation-in-part of application No. 13/261,391, filed on Aug. 1, 2012, now abandoned.
(60) Provisional application No. 61/296,761, filed on Jan. 20, 2010.

(51) Int. Cl. A61B 17/88; A61B 17/86; A61B 5/107

(52) U.S. Cl. CPC A61B 17/8811; A61B 5/1076; A61B 17/861

(57) ABSTRACT

an orthopedic tube for delivering medical fluids, having a viscosity of at least 1 Pa.S and not to exceed 10000 Pa.S, into a bone cavity comprising an elongated hollow stem having a smooth stem outer wall. The elongated hollow stem has an external diameter sufficient to be inserted into a bone cavity formed in a bone. The elongated hollow stem has a proximal end and a distal end with the proximal end adapted to be in communication with a pressurized medical fluid injector; and the elongated hollow stem has an internal diameter sufficiently large to permit a path of least resistance to a flow of the cement from the injector. The distal end of the elongated stem is closed. There is at least a fenestration zone defined on the stem wall spaced from the distal end, and a plurality of distinct ports distributed in a pattern in the fenestration zone. The diameter of the ports in the fenestration zone determines the fluid dispersion pattern and the distribution of ports relative to the internal diameter of the elongated hollow stem is such that the fluid will fill up the hollow stem first until sufficient pressure is built up to seep the fluid through the ports in a uniform manner. Am method of measuring the density of the bone is also described.

### CROSS REFERENCE TO RELATED APPLICATIONS

This application is a Continuation-in-Part of co-pending application Ser. No. 13/261,391, filed on 1 Aug. 2012, for which priority is claimed under 35 U.S.C. §120; which application claims priority of International Application No. PCT/CA2011/000073 filed on 20 Jan. 2011 under 35 U.S.C. §119; and which also claims priority of U.S. Provisional Application No. 61/296,761 filed on 20 Jan. 2010 under 35 U.S.C. §119(e), the entire contents of all of which are hereby incorporated by reference.

### FIELD OF THE INVENTION

The present invention relates to minimally invasive methods and devices for orthopedic procedures and applications to other body sites.

### BACKGROUND ART

There are many applications where a minimally invasive injection procedure may be used to treat various body lesions. Although orthopedics, has for us, captured the attention of biomedical developments involving minimally invasive catheters and the like, we understand that similar procedures may be used to treat other body lesions, as will be discussed.

With an aging population it is important to improve the screening of osteoporosis. Osteoporosis is a degenerative skeletal disease that is characterized by reduced bone strength and exposes patients to a greater risk of fracture most commonly at the spine, wrist or hip. There are over three hundred million women/men taking drugs and medication for osteoporosis. The conventional method of measuring the quality of bone is to indirectly measure bone density. Physicians do not have an objective method of measuring the bone quality prior to, or during a procedure. Bone quality is being mainly measured through imaging techniques and these measurements are related to the density of the bone. The density of the bone is indicative of the mechanical strength thereof, but not always. There are some cases that show a dense bone that is otherwise mechanically weak.

Surgeons often rely on a DXA scan of the patient. However, in practice more than 50% of the procedures are conducted without bone scans. For the remainder of the procedures physicians must rely on their own experience making decisions based on subjective data.

Another problem that has been identified is the distribution of the cement within the cavity formed within the bone. Although catheters have been designed with stems defining side ports for distributing the cement around the vicinity of the catheter, the cement tends to exit in an uneven fashion. This results in an uneven distribution of the cement within the bone cavity.

It has also been found that when repairing damaged bones with screws, physicians will perform a vertebroplasty in order to ensure that a pedicle screw will be properly fixed. But such a procedure is excessive. As a result there are available canulated pedicle screws to allow physicians to submit cement through the screws once the screws have been placed in the pedicle. This canulated screw includes a central bore with side fenestrations to allow the cement to be distributed around the threads of the screw. However it has been found that such techniques lead to significant leakage while not providing sufficient cement to anchor the pedicle screw.

Accordingly, improvements are desirable.

### SUMMARY

It is therefore an aim of the present invention to provide an improved minimally invasive device and related methods.

It is a further aim of the present invention to provide a device for distributing a medical fluid into a body site with uniform distribution of the fluid into the body site.

It is a still further aim of the present invention to provide improved orthopedic devices for testing bone density and also more uniform distribution of cement in bone sites.

In accordance with one aspect, there is a medical catheter for delivering medical fluids, having a viscosity of at least 1 Pa.S and not to exceed 10000 Pa.S, into a body site comprising an elongated hollow stem having a smooth stem outer wall. The elongated hollow stem has an external diameter sufficient to be inserted into a body site at a body lesion. The elongated hollow stem has a proximal end and a distal end with the proximal end adapted to be in communication with a pressurized medical fluid injector; and the elongated hollow stem has an internal diameter sufficiently large to permit a path of least resistance to a flow of the medical fluid from the injector. The distal end of the elongated stem is closed. There is at least a fenestration zone defined on the stem wall spaced from the distal end, and a plurality of distinct ports distributed in a pattern in the fenestration zone. The area of each port in the fenestration zone determines the fluid dispersion pattern and the distribution of ports relative to the internal cross-sectional area of the elongated hollow stem is such that the fluid will fill up the hollow stem first until sufficient pressure is built up to seep the fluid through the ports in a uniform manner.

In a more specific embodiment, the ports may have gradually increased areas as the distance from the distal end increases.

The fluid may be a gel or medical cement. The fluid may include biodegradable polymers. Such biodegradable polymers may be used for injecting regenerative cells for curing body lesions found in the heart, eyes and other body sites.

In another aspect, there is a method of injecting a fluid having a viscosity of between 1 Pa.S and 10,000 Pa.S, into a site of a body lesion, including providing an elongated tube having a closed distal end and a plurality of ports in a fenestration zone on the tube wall wherein the fluid is delivered under pressure into the tube from the proximal end thereof to fill the tube while restricting the fluid from exiting through the ports based on the relative viscosity of the fluid; continuing to apply pressure on the fluid once the tube is filled to simultaneously overcome the resistance at the ports allowing the fluid to exit the ports in an uniform manner into the body site.

In another aspect, there is provided an orthopedic device comprising a hollow cannula having a distal end including an engagement member for engaging a bone, an elongated probe extending through the hollow cannula, and a metering device moving the probe such that a distal end of the probe is moved away from a proximal end of the hollow cannula to extend beyond the distal end of the hollow cannula, the metering device measuring a force applied by the distal end of the probe.

Also in accordance with a further aspect, there is provided a method of measuring a strength of a bone, comprising engaging a distal end of a hollow cannula with the bone, inserting an elongated probe into the hollow cannula, advancing the elongated probe into the hollow cannula until the elongated probe penetrates the bone, and while penetrating the bone with the elongated probe, measuring a force applied by the elongated probe on the bone as an indication of the strength thereof

Also in accordance with still another aspect, there is provided a method of consolidating a bone, comprising engaging a distal end of a hollow cannula with the bone, inserting an indenter into the hollow cannula and penetrating the bone with the indenter beyond the hollow cannula, removing the indenter from the hollow cannula, leaving a channel defined in the bone, engaging a screw into the channel, and retaining the screw within the channel with bone cement.

Further in accordance with the present invention, there is provided a bone screw comprising a head connectable to a cement delivery device, and a hollow stem extending from the head and in fluid communication therewith, the hollow stem being defined by a tubular wall including threads on an exterior surface thereof, the tubular wall including lateral ports defined therethrough along at least a majority of a length of the hollow stem and closed at a distal end.

When the term catheter is used in the specification it may also represent a needle.

### BRIEF DESCRIPTION OF THE DRAWINGS

Reference will now be made to the accompanying drawings, showing by way of illustration a particular embodiment of the present invention and in which:

FIG. 1 is a perspective view of a typical vertebra;

FIG. 2 is a schematic lateral view of a spinal segment showing a fractured vertebra;

FIG. 3 is a top view of a threaded cannula being placed in a typical vertebra shown in horizontal cross-section;

FIG. 4 is a schematic top view a handheld metering device mounted to the indenter shown advanced in the vertebra;

FIGS. 5a and 5b are side views of the detail of a cement injector;

FIG. 6 is a view showing the cement injector inserted into the channel in the vertebra by means of the cannula and showing cement seeping out of the distal end of the cannula;

FIG. 7a is a view of the vertebra with the cannula removed and the pedicle screw being inserted into the channel formed in the bone;

FIG. 7b is a view similar to FIG. 7a but showing the pedicle screw anchored in the vertebra;

FIG. 8 is a fragmentary view of a pedicle screw in accordance with another embodiment;

FIG. 9 is a schematic lateral view showing two vertebrae fixed by pedicle screws and a bridge rod;

FIG. 10 is a lateral fragmentary schematic view showing a channel, in the neck of the femoral bone, formed by the threaded cannula and an indenter;

FIGS. 11a and 11b are lateral fragmentary schematic views showing the details of the invention of FIG. 10 with the cement injector in different operative positions;

FIG. 12 is a lateral fragmentary schematic view showing the neck of a femoral bone using a dynamic hip screw in an attempt to repair an intertrochanteric fracture with the use of cement to anchor the dynamic hip screw.

FIGS. 13 is a schematic view of a hollow stem as shown in FIGS. 5a, 5b, and 8;

FIGS. 14a,14b, 14c and 14d are schematic views showing the filling of a tube as shown in FIGS. 13 with cement;

FIG. 15 is a graph representing the progress of the filling of the tube with cement and the distribution of the cement; and

FIG. 16 is an image representing, in real-time, the distribution of the cement from the stem.

### DETAILED DESCRIPTION OF PARTICULAR EMBODIMENTS

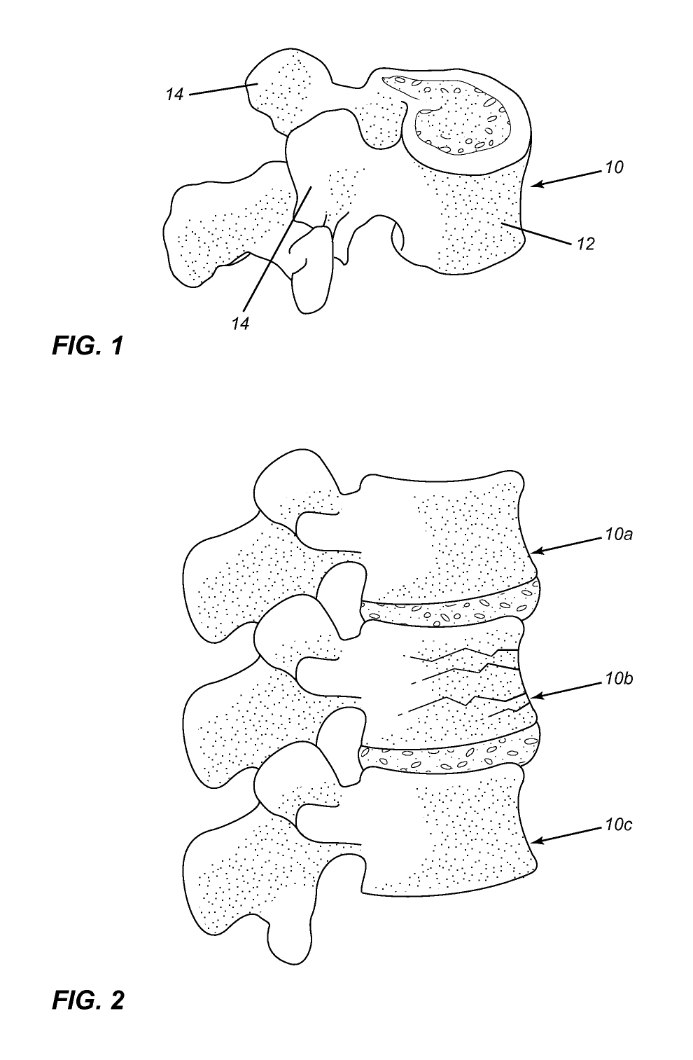

Referring now to FIGS. 1 a vertebra 10 is illustrated having a vertebral body 12 and a pedicle 14. In FIG. 2 a fractured vertebra 10b is illustrated within a spinal segment consisting of vertebrae 10a, 10b and 10c with vertebra 10b shown damaged. The medical condition shown in FIGS. 2 may be corrected by using a spinal fixation as will be described.

The following is a description of one embodiment of the present invention as it applies to the spine. It is understood that the device described in the present embodiment can be utilized to repair other bone structures in the body as will be described further below, for instance in pelvis or in the hip.

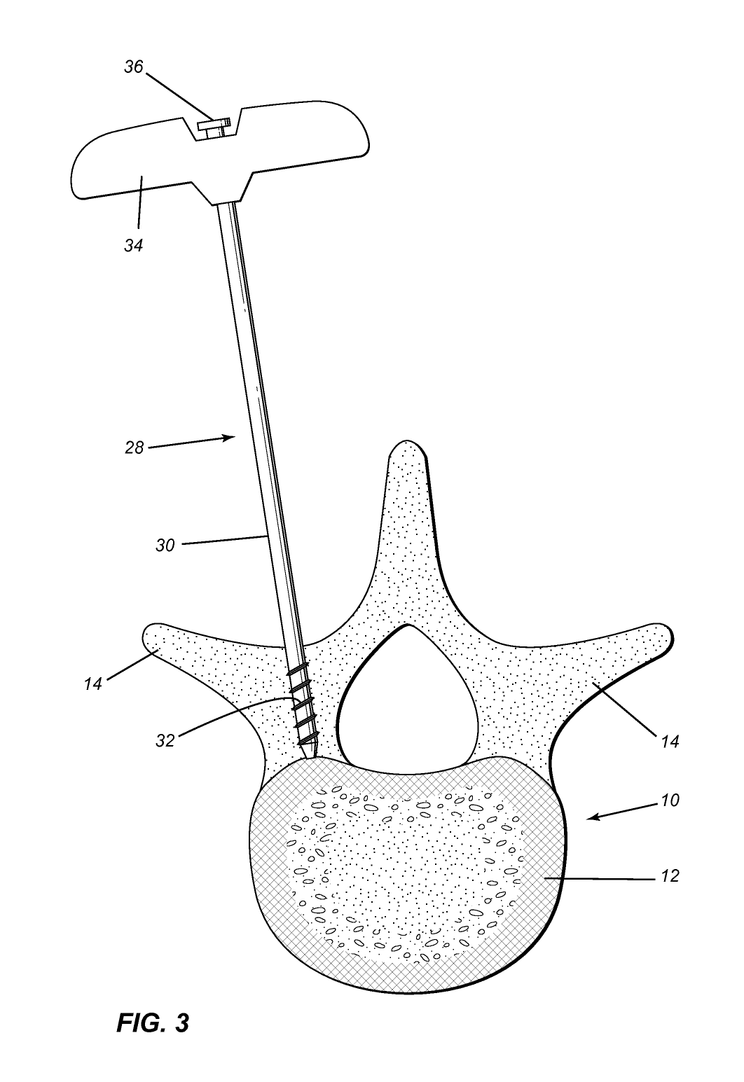

FIG. 3 illustrates a cannula 28 including a hollow stem 30 having tapping threads 32 on its distal end for engaging the bone. The proximal end of the cannula 28 includes a handle 34 and a luer adapter 36. The cannula 28 is shown being inserted into the pedicle 14 of a typical vertebra 10 to be repaired. The cannula 28 may be provided with a stylet (not shown) to ease the piercing of soft tissues and bone.

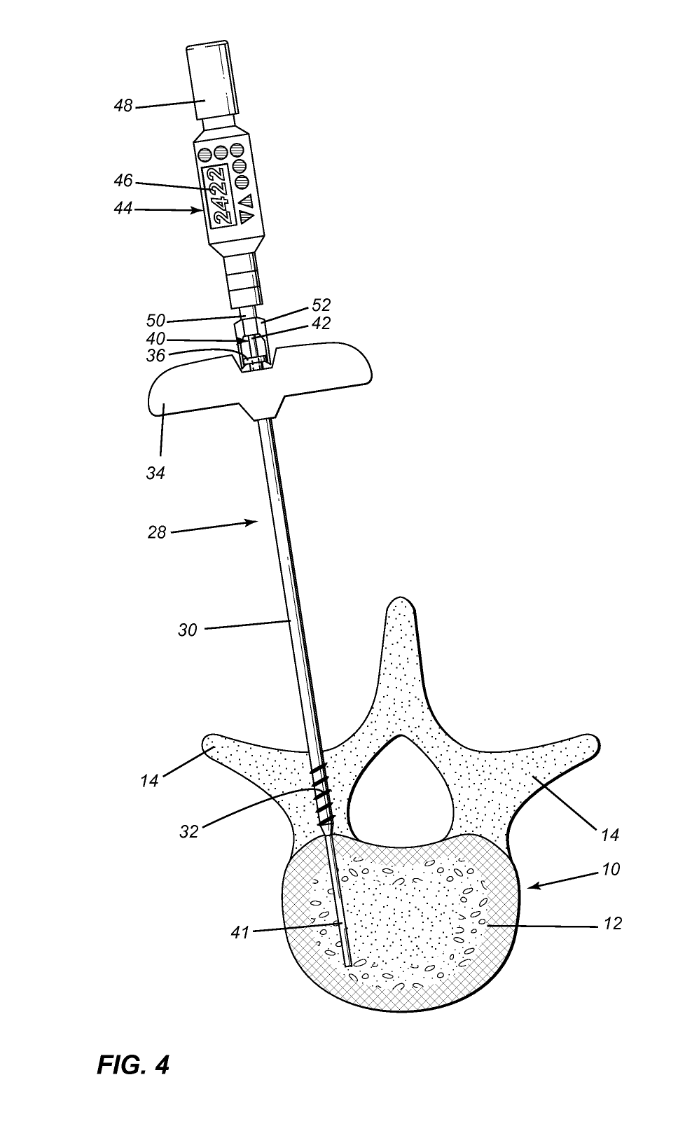

FIG. 4 shows the use of a probe or indenter 40 having a distal end 41 and a proximal end 42. The indenter 40 is a rod that can pass through the bore formed in the stem 30 of the cannula 28. The purpose of the indenter 40 is to allow an evaluation of the quality of the bone. For instance, by applying a linear force on the indenter 40 into the vertebral body the strength of the bone can be measured by measuring the force required to advance the indenter 40 in the bone structure of the vertebral body 12. Also shown is a metering device 44 includes a motor (not shown) which provides the force required to advance the indenter 40 into the bone structure. A load cell (not shown) provides the data representing the quality of the bone and the data is displayed on the display 46. The metering device 44 includes a handle 48, and a coupling device 50 adapted to be connected to the indenter 40. Attachment devices 52 (not shown) may be provided to connect the metering device 44 to the luer 36 on the cannula 28.

The load cell measures the force as the indenter 40 advances into the vertebra 10 by measuring the reaction force directly on the indenter 40. Alternatively the force may be measured from the current required to run the motor at a constant speed to advance the indentor 40 at a constant velocity over a predetermined distance. In one embodiment, the distance to be traveled is from 3 to 5 cm while the speed was determined to be 2.5 cm per second. The indenter 40 and metering device 44 thus provide an instant and objective measurement of the bone resistance to the compressive force applied by the indenter 40, thus allowing a direct measure of the hardness and strength of the bone. Since the cannula 28 is anchored to the bone by means of the threads 32 and the metering device 44 is fixedly coupled to the cannula 28, the device provides the necessary support for the linear progress of the indenter 40 by resisting the reaction force thereon.

In a particular embodiment, at least the indenter 40 is disposable. A solid embodiment of the indenter 40 is shown. Alternately the indenter 40 may be hollow. If the stem of the indenter 40 is hollow it can be used to collect a core sample of the bone for use in a biopsy examination.

Advantageously, the bone strength measurement can be done prior to surgery. The advantage of the device is also that it can be used in the examination room for mass screening and follow up on therapeutic treatment. These measurements can be done under local anesthesia and in outpatient clinics for screening of osteoporotic patients.

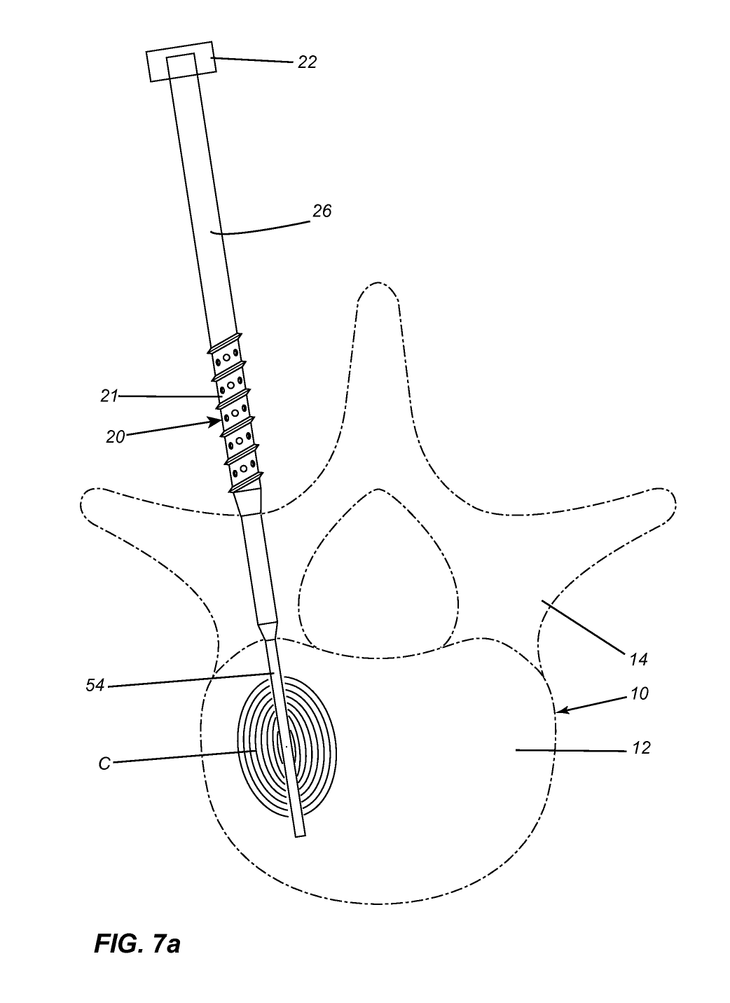

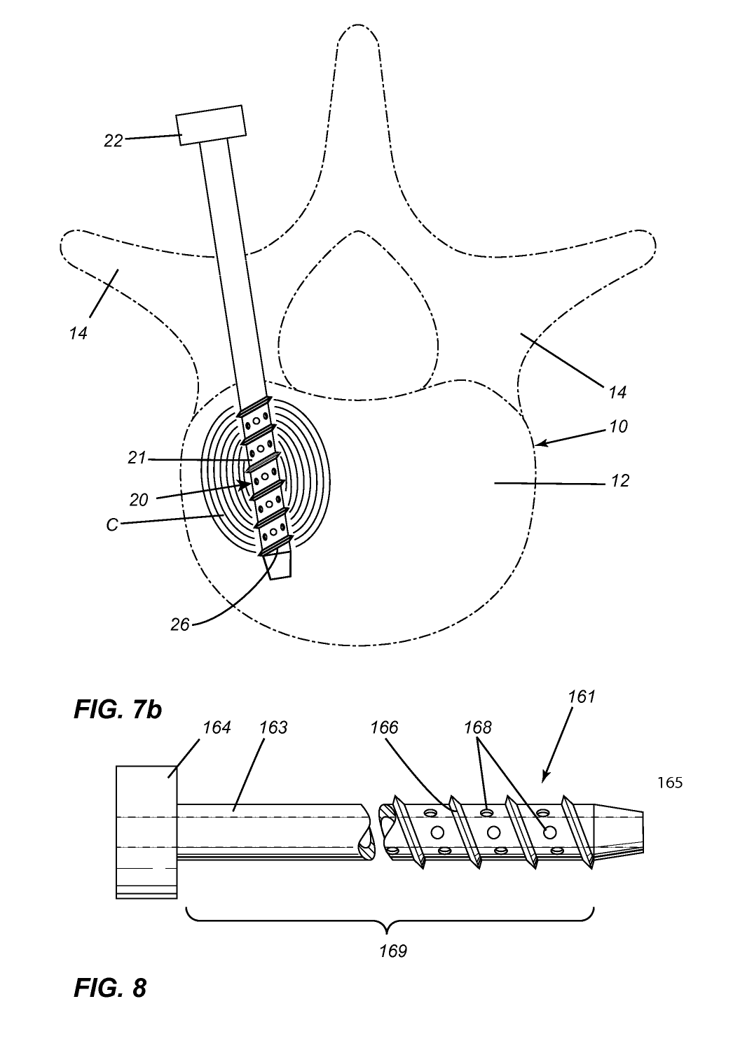

The cannula 28 and indenter 40 can also be used to prepare for the insertion of a bone screw. Once the indenter 40 has been inserted into the body 12 of the vertebra 10 and the quality of the bone structure has been measured, the indenter 40 is withdrawn from the cannula, leaving behind a channel 54 (see FIG. 7a) in the vertebral body 12.

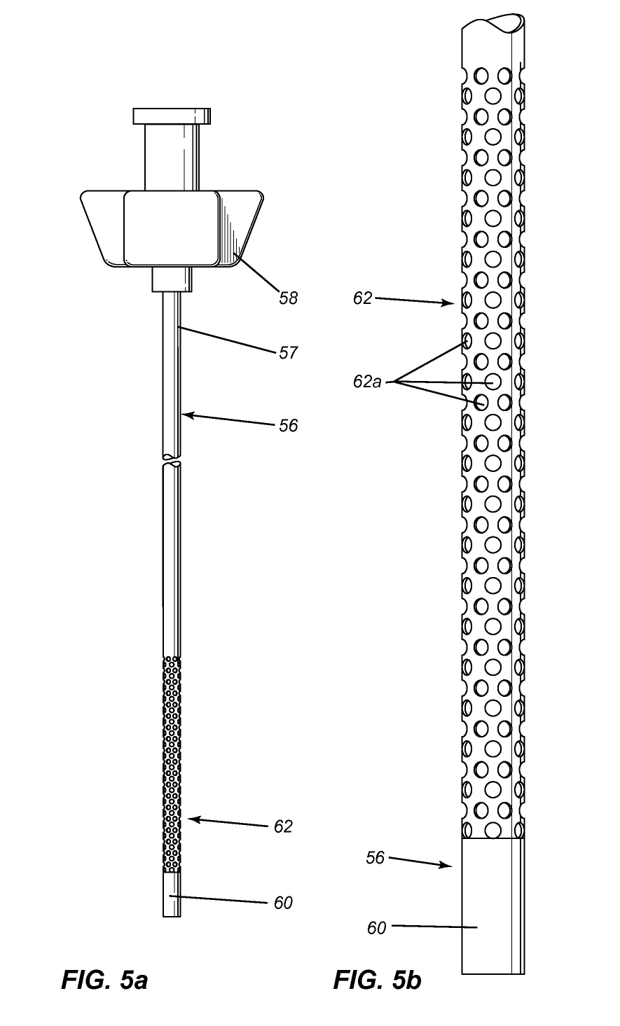

FIG. 5a illustrates a cement delivery tube 56 including a hollow stem 57 and a luer adapter 58 at its proximal end. The diameter of the stem 57 is such that it can be easily inserted into the hollow stem 30 of the cannula 28. FIG. 5b shows an enlarged view of the stem 57 of the cement delivery tube 56 with a fenestration zone 62 made up of a pattern of ports 62a-62e. The ports 62a-62e are consistently distributed throughout the fenestration zone 62. To allow a uniform dispersion of the cement when it is forced through the cement delivery tube 56. In the embodiment shown, the distal end 60 of the cement delivery tube 56 is blocked. The inner diameter of the cement delivery tube 56 should be sufficiently large as to form a path of least resistance for the cement so that the cement will fill up the tube 56 before leaking through the ports 62a-62e. Once the tube 56 is filled with cement, the pressure is built up within the delivery tube 56 and then the cement will flow out of the tube 56 around the fenestration zone 62 in a uniform manner. The length of the fenestration zone 62 can be varied to thus control the pressure drop. Furthermore the shape, diameter, and number of ports 62a-62a in the fenestration zone 62 can be varied in order to control the cement dispersion pattern and its uniformity.

For instance the ports 62a may be gradually increased in diameter towards ports 62e to compensate for the slightly reduced pressure as the distance from the end 60 increases, in order to overcome the resistance caused by the viscosity of the cement or other fluid.

We have discovered that the smaller the cross-sectional area of bore or the cannulation of tube 56 the higher the pressure drop inside the tube. However the larger the area of the port 62a, the easier it will be for the viscous material to seep through the ports 62a as the material flows into the tube 56. The smaller the ratio of the port area to the cannulation area the greater is the uniformity of the distribution as cement, gel or other fluid exits from the tube 56.

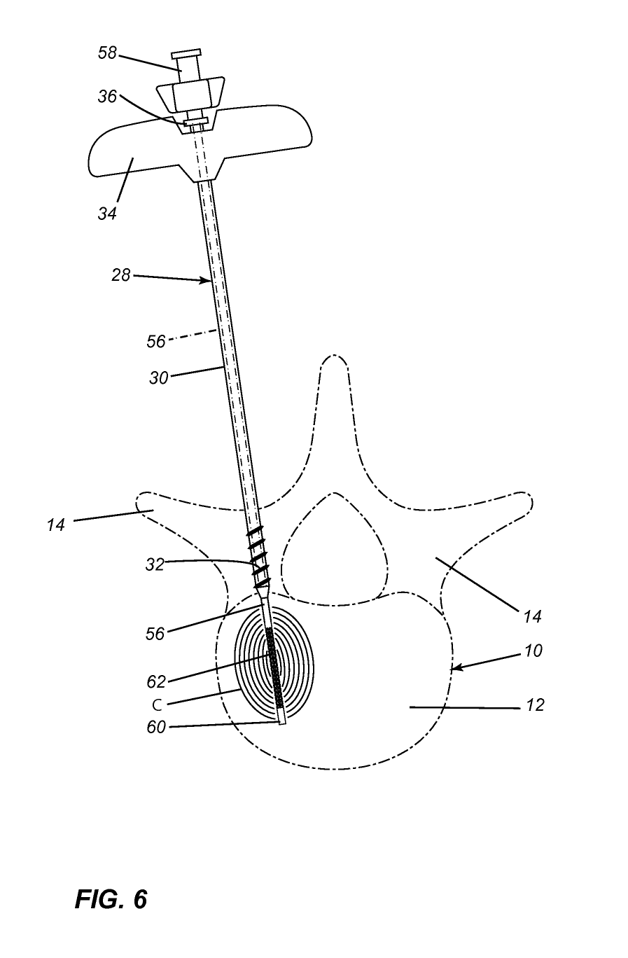

As can be seen in FIG. 6, the cement delivery tube 56 is inserted into the vertebral body 12 through the cannula 28. The distal end 60 of the cement delivery tube 56 extends beyond the distal end of the cannula 28 into the channel 54 formed in the bone structure of the vertebral body 12 by the indenter. The fenestration zone 62 extends within the channel 54. The luer adapter 58 of the cement delivery tube 56 is connected to a cement delivery device (not shown). The delivery tube 56 may be primed with cement prior to being inserted into the vertebral body 12, or may be simply inserted through the cannula 28 before cement is fed thereinto. FIG. 6 shows, schematically, the dispersion pattern of the cement after it has been injected through the cement delivery tube 56. The cement delivery tube 56 could be flexible with memory such that it can be curved to extend in a lateral direction once it has extended beyond the distal end of the cannula 28.

Once the cement has been injected and is curing, but not yet set, the next step involves removing the cannula 28 from the vertebra 10 and inserting a pedicle screw 20 into the channel 54 as shown in FIGS. 7a and 7b. The stem 21 of the pedicle screw 20 is slightly larger than the channel 54 so that the tapping threads 26 of the pedicle screw positively engage the bone structure as well as the cement C dispersed in the cavity formed in the vertebral body 12. The pedicle screw 20 must be inserted into the channel 54 prior to the cement C being completely cured, and the cement will set on the threads of the screw 20 in order to anchor it. The procedure including the use of the cannula 28 and the cement delivery tube 56 provides a prepared site for receiving the pedicle screw 20.

FIG. 8 shows another embodiment of the pedicle screw. In this embodiment, the pedicle screw 161 includes a stem 163 which is hollow. The pedicle screw 161 also includes a head 164 connectable to a cement delivery device (not shown) and spirals threads 166 on the exterior surface of the hollow stem 163. Furthermore the hollow stem 163 includes a large number of lateral ports 168 disposed between the threads 166 and providing a fenestration zone 169 extending along a majority of the length of the pedicle screw 161, and in the embodiment shown, extending along the entire length of the screw 161. As can be seen, the pedicle screw 161 can be used as an alternative to the cement delivery tube 56. The distal end 165 of pedicle screw is closed. Thus, as the cement is forced, under pressure, into the hollow stem 163 of the screw 161, it will first fill the hollow stem 163 whereby the pressure will increase until the cement will start to flow uniformly through the lateral ports 168.

In this embodiment, the cannula 28 is removed once the bone quality diagnostic procedure with the indenter 40 is terminated. The channel 54 left by the cannula 28 and the indenter 40 serves to receive the pedicle screw 161. Once the pedicle screw 161 is in place within the channel 54, cement can be delivered through the hollow stem 163.

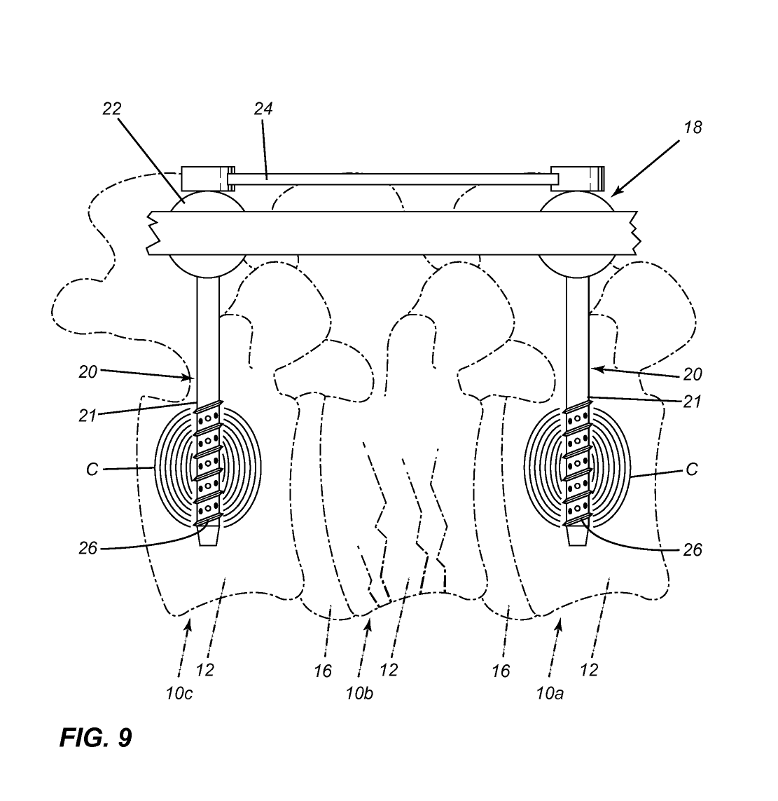

FIG. 9 shows a typical spinal fixation 18 but using screws as described in the present specification. The fixation 18 includes pedicle screws 20 made up of a screw stem 21, head 22 and spirals threads 26 on the stem 21. A bridge rod 24 is fixedly connected to the pedicle screw heads 22 in order to provide a proper stabilization structure between two healthy vertebra bodies 10a and 10c. Thus the fractured vertebra is protected against motion occurring at the disc or vertebra 10b.

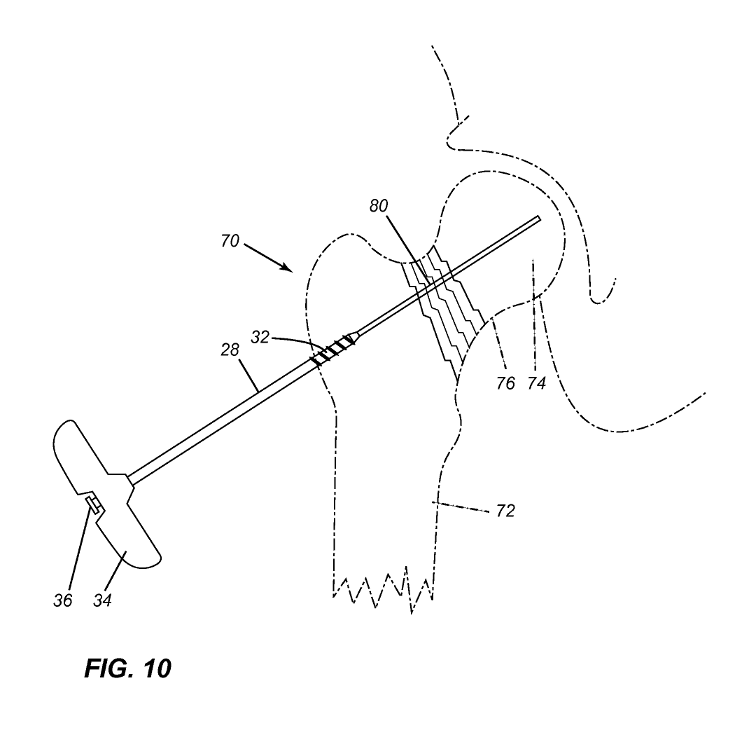

In another application of the present invention, there is shown in FIG. 10 a typical hip 70. The hip 70 includes a femoral bone 72 having a femoral head 74 and a femoral neck 76. The femoral head 74 fits into the pelvis 78 as is well known. A fracture at the femoral neck 76 and an intertrochanteric fracture are shown.

Similarly to the previously described application, the cannula 28 is inserted into the femoral bone 72 coaxial with the femoral neck 76, as shown in FIG. 10. The threads 32 of the cannula 28 engage the bone as it is being inserted. The indenter 40 is inserted through the hollow stem 30 of the cannula 28 and is forced under pressure into the bone structure of the femoral neck 76 and into the head 74 for the purposes of measuring the quality of the bone structure through the use of the metering device 44.

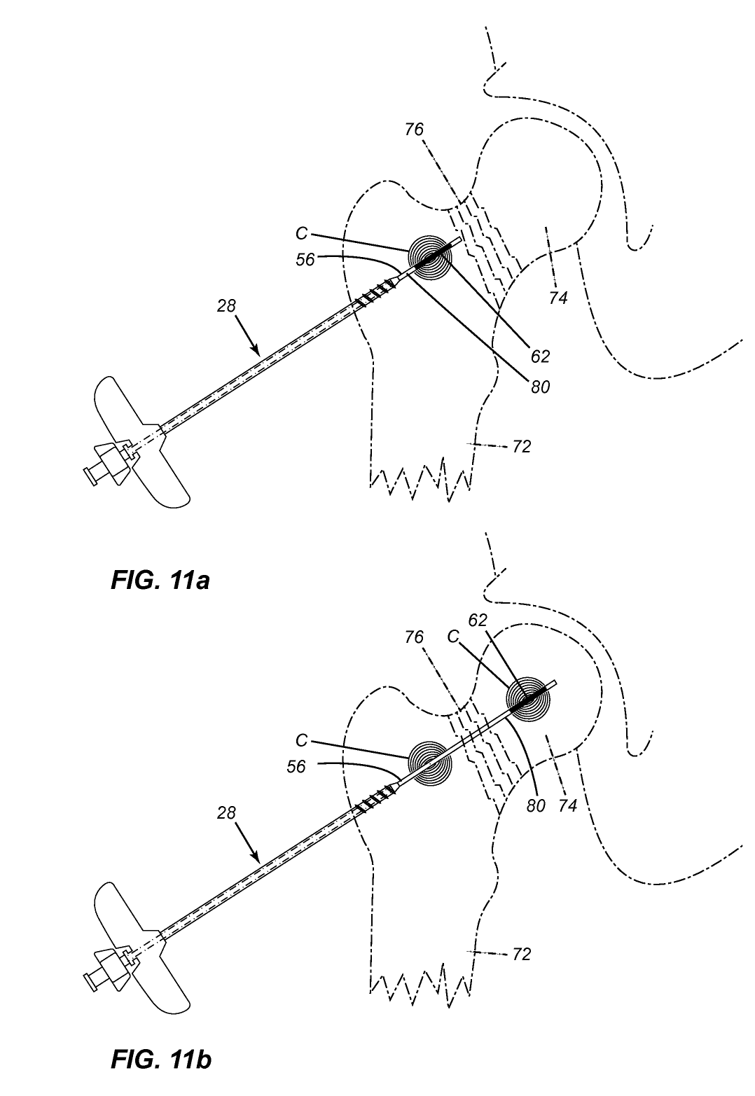

The indenter 40 once removed from the cannula 28 leaves a channel 80 in the femoral neck 76 and head 74. As shown in FIG. 11a, the cement delivery tube 56, is then inserted through the hollow stem 30 of the cannula 28 into the channel 80. As previously described cement is then fed under pressure into the cement delivery tube 56, and cement is expelled uniformly around the fenestration zone 62. FIG. 11b shows a different application of the cement delivery. In this embodiment the fenestrations zone 62 could be located more toward the middle of the tube 56 or at the distal end. The cement can thus de disposed as shown.

The location of the cement C in different locations such as in the femoral head 74 or in the femoral neck 76. As previously described, the design of the fenestration zone 62, whether at the distal end of the delivery tube 56 or in the middle of the delivery tube 56, dictates the location of the cement C which flows through the tubes 56, with relative ease under pressure to be dispersed uniformly around the fenestration zone 62, wherever it is located.

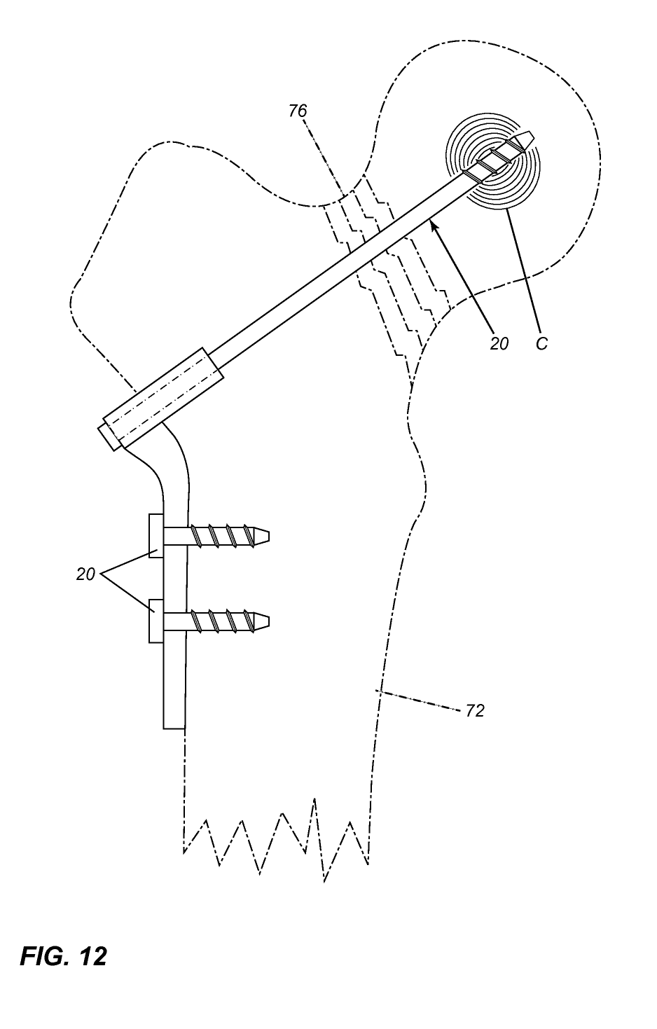

Different applications of screws 20 to repair fractures in the femoral neck 76 or the femoral head 74 may be contemplated. For instance in FIG. 12, a dynamic hip screw 20 is shown, which is similar to the pedicle screw 20 previously described. In all applications the threads on the pedicle screws are cemented as previously described. Alternately, hollow screws 161 similar to those previously described may be used, such that the cement may be injected directly therethrough.

As previously described, it is advantageous to provide a uniform delivery of the cement, gel or other fluid, to be delivered through the elongated stem 56, having a fenestration zone 62, into the bone cavity or similar body site. Thus, the uniform distribution does not depend on the number of ports 62a-62e provided in the fenestration zone cement or gel, but on the relatively small ratio of the area of the cross section of the tube 56 and the area of the individual ports 62a-62e.

The following examples are based on the feature of a uniform distribution of cement or gel described above in relation to FIGS. 5a and 5b.

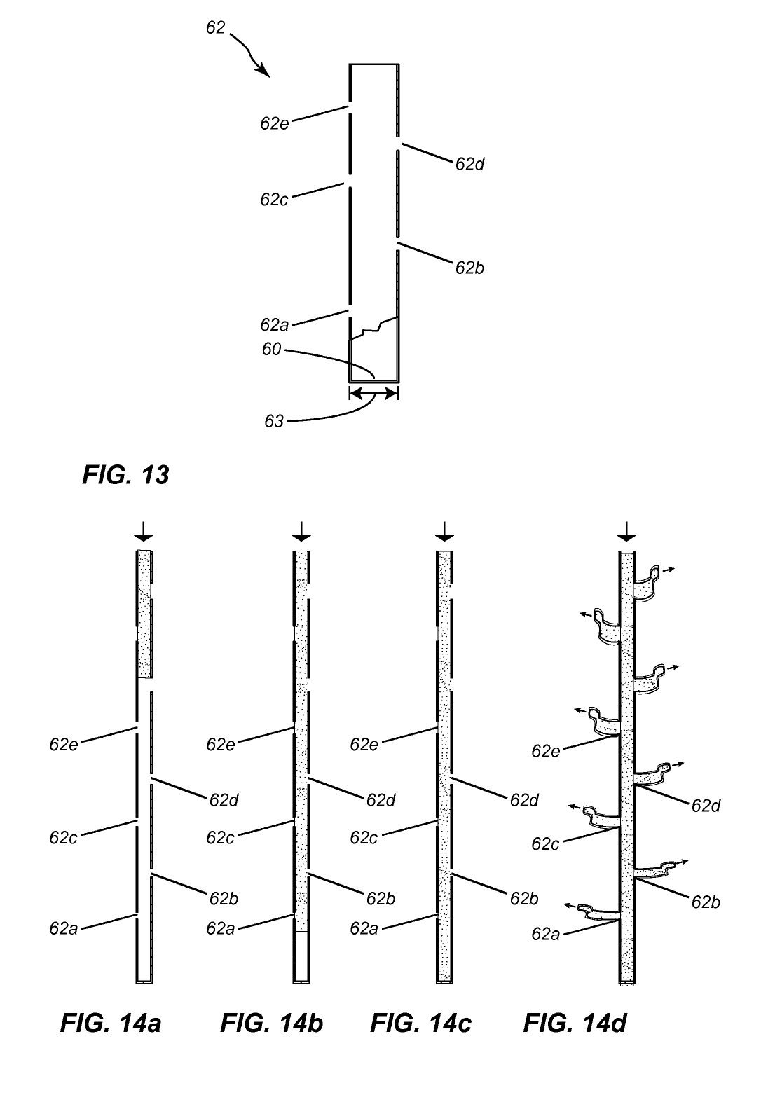

Example I is as shown in FIG. 13:

Viscosity of the cement:
1,000 to 5,000 Pa.S

Internal diameter 63 of the stem 56 in the fenestration zone 62:
2.25 mm
diameter of a port 62a: 0.3 mm
diameter of a port 62b: 0.35 mm
diameter of a port 62c: 0.4 mm
diameter of a port 62d: 0.45 mm
diameter of a port 62e: 0.5 mm
wall thickness of the stem 56:

Thus the internal area of the stem 56 in the fenestration zone 62 is 3.97 mm² while the area of a port 62a is 0.07 mm²

In another example port 62a had a diameter of 0.25 mm. The other rows of ports 62b-62e increased proportionally.

Thus the internal area of the stem 56 in the fenestration zone 62 is 3.97 mm² while the area of a port 62a is 0.05 mm².

The viscosity of the fluid such as medical cement or even a gel will impact on the parameters of the internal diameter 63 of the stem 56 in the fenestration zone 62. For instance in the case of a gel the viscosity typically will be in the range of 1 Pa.S to 10 Pa.S. Thus it is contemplated that the hollow stem 56 would have a cross-sectional area in the range of between 1 mm² to 4 mm². In this case the port size would be in the range of 0.01 mm² to 0.05 mm².

However in the case of medical cement where the viscosity can be in the range of 100 to 10,000 Pa.S, the parameters of the hollow stem will be greater. For instance, the cross-sectional area of the hollow stem 56 might be in a range of between 3 to 15 mm² while the port area would be in a range of 0.05 to 3 mm².

The pedicle screw shown in FIG. 8 may be constructed in accordance with the parameters of the above embodiments to accommodate medical cement. The screw would be provided with a closed distal end.

The phenomena are illustrated in the series of schematic illustrations shown in FIGS. 14a to 14d. The cement enters the proximal end of the tube 56 in FIG. 14a (cement is shown as the shaded portion). As the cement progresses to fill the tube 56 in FIGS. 14b-14c it is prevented from seeping through ports 62a in fenestration zone 62 because of the size of the ports 62a relative to the viscosity of the cement. Once the cement fills the tube 56 and pressure continues to be applied to the cement, it will exit the tube, in a uniform manner, as illustrated in FIG. 14d.

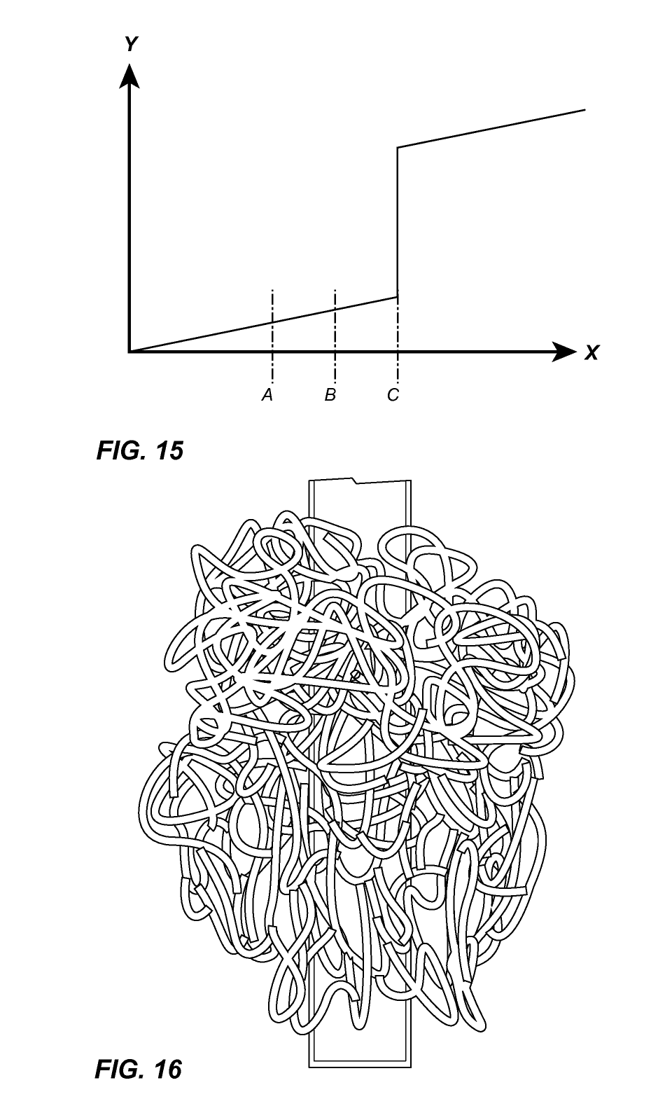

FIG. 15 represents the progression of the cement within the tube 56 where the“x” axis is the time of application and the “y” axis is the pressure applied to the cement. Positions “A”, “B” and “C” on axis “x” represent the time lapse shown in FIGS. 14a, 14b and 14c, 14d.

It has been determined that the ports 62a-62e may be of progressively increasing diameter (and thus area) as the rows are farther from the distal end 60. As one moves distally along the stem 56, the pressure decreases slightly and because of the viscous nature of the fluid, the ports are increased to compensate for the resistance to flow thereof. It means that the first row of ports 62a is subject to a higher pressure than the rows of ports 62b-62e that are more distant from the distal end 60. To provide for a uniform feed of the fluid from all ports, and to compensate for the pressure reduction/loss as one moves more distant from the end 60, the diameter of the ports can be increased, from 10 to 20 percent.

FIG. 16 shows the cement exiting the tube 56, in real time.

The examples provided above are based on the use of cement in musculoskeletal tissue. However the stem 56 could be used for injecting polymers and cell generating materials into other parts of the body to treat lesions where catheters or needles would be used to inject such material. For instance the stem could replace traditional needles used for injecting therapeutic viscous materials into arteriovenous malformations; lesions in the eye; the muscular tissue of the heart for example after an infarction. Recent studies have promoted the use of injectable polymers, in some cases, to treat such lesions. The important feature of the cannula stem 56 in these embodiments and applications is the uniform distribution of the medical viscous material.

The embodiments of the invention described above are intended to be exemplary. Those skilled in the art will therefore appreciate that the foregoing description is illustrative only, and that various alternate configurations and modifications can be devised without departing from the spirit of the present invention. Accordingly, the present invention is intended to embrace all such alternate configurations, modifications and variances which fall within the scope of the appended claims.

1. A medical catheter for delivering medical fluids which have a viscosity of at least 1 Pa.S and less than 10000 Pa.S; into a body site comprising an elongated hollow stem having a smooth stem outer wall, the elongated hollow stem having an external diameter sufficient to be inserted into a body site at a body lesion; the elongated hollow stern having a proximal end and a distal end with the proximal end adapted to be in communication with a pressurized medical fluid injector; the elongated hollow stem having an internal cross-sectional area sufficiently large to permit a path of least resistance to a flow of the fluid from the injector, the distal end of the elongated stem being closed; at least a fenestration zone defined on the stem wall spaced from the distal end of the hollow stem; a plurality of distinct ports distributed in a pattern throughout the fenestration zone; the area of each port relative to the internal cross sectional area of the elongated hollow stem is such that the fluid will fill up the hollow stem first until sufficient pressure is built up to seep the fluid through the ports in a uniform manner.

2. The catheter as defined in claim I wherein the internal cross-sectional area of the hollow stem is in the range of 1 mm² to 15 mm² while the cross-sectional area of each port is in the range 0.01 mm² to 3 mm².

3. The catheter as defined in claim 2 including an orthopedic stem wherein the medical fluid is a medical cement having a viscosity in the range of 100 to 10,000 Pa. S, the cross-section of the hollow stem will he in the range of 3 to 15 mm² and the area of a port will be in the range of 0.05 mm² to 3 mm².

4. The orthopedic stem as defined in claim 3 wherein the medical cement has a viscosity of 1,000 to 5,000 Pa.S, the cross-sectional area of the hollow stem is 3.97 mm² while the area of a port closest to the distal end is 0.03 mm² progressing to 0.05 mm² at a distance from the distal end.

5. The catheter as defined in claim 2 wherein the medical fluid is a gel having a viscosity in the range of 1 Pa.S to 10 Pa.S the cross section of the hollow stem will be in the range of 1 mm² to 4 mm² and the port area size will be in the range of 0.01 mm² to 0.05 mm².

6. The catheter as defined in claim 5 wherein the fluid is a therapeutic polymer.

7. The catheter as defined in claim 1 wherein the catheter is a needle.

8. The catheter as defined in claim 3 where the catheter is a screw having threads on the outer smooth wall and the ports are located between the thread pitch.

9. A method of injecting a fluid having a viscosity of between 1 Pa.S and 10,000 Pa.S, into a site of a body lesion, including providing an elongated tube having a closed distal end and a plurality of ports in a fenestration zone on the tube wall wherein the fluid is delivered under pressure into the tube from the proximal end thereof to fill the tube while restricting the fluid from exiting through the ports as a function of the relative viscosity of the fluid; continuing to apply pressure on the fluid once the tube is filled to simultaneously overcome the resistance at the ports allowing the fluid to exit the ports in an uniform manner into the body site.

10. An orthopedic kit for determining the mechanical strength of bone comprising:
a hollow cannula including an engagement member, at least at a distal end thereof, for engaging a bone;
an elongated probe to be passed through the hollow cannula and having a length sufficient to extend beyond the distal end of the cannula;
a motor device mounted in a housing attachable to the cannula with the motor device connectable to the probe to drive the probe such that a distal end of the probe is moved away from the distal end of the hollow cannula to extend into the bone; and
a metering device for measuring a force applied by the probe to the bone.

11. A method of measuring the mechanical strength of a bone, comprising:
engaging a distal end of a hollow cannula with the bone;
inserting an elongated probe into the hollow cannula;
advancing the elongated probe into the hollow cannula until the elongated probe penetrates the bone; and
while penetrating the bone with the elongated probe, measuring a force applied by the elongated probe on the bone as an indication of the mechanical strength thereof.

12. The method as defined in claim 14 wherein the cannula engages the bone in order to anchor the cannula to the bone and to resist to the reactive forces of the probe being driven in the bone.

13. A method of consolidating a bone, comprising:
engaging a distal end of a hollow cannula with the bone;
inserting an indenter into the hollow cannula and penetrating the bone with the indenter beyond the distal end of the hollow cannula;
removing the indenter from the hollow cannula, thus leaving a channel defined in the bone;
engaging a screw into the channel; and
retaining the screw within the channel with bone cement.

14. A bone screw comprising a head connectable to a cement delivery device, and a hollow stem extending from the head and in fluid communication therewith, the hollow stem being defined by a tubular wall including threads on an exterior surface thereof, the tubular wall including lateral ports defined there through along at least a majority of a length of the hollow stem.

15. An orthopedic tube for delivering medical cement into a bone cavity comprising an elongated hollow stem wall, having an external diameter sufficient to be inserted into an injection conduit formed in a bone and an internal diameter sufficiently large to permit a path of least resistance to the cement flow, terminating in a closed distal end; at least a fenestration zone defined on the stem wall spaced from the distal end; a pattern of ports distributed throughout the fenestration zone; the distribution of the ports in the fenestration zone relative to the internal diameter of the hollow stem wall such that the cement will fill up the hollow stem first until sufficient pressure is built up to seep the cement through the ports in a uniform manner.

16. A method of injecting medical cement into a bone including the steps of forming an injection conduit in the bone; inserting an orthopedic tube in the conduit with the tube having a fenestration zone made up of a pattern of ports distributed throughout the fenestration zone; filling the tube with cement applying pressure on the cement to cause the cement to be injected into the bone through the ports in a uniform manner.
